(19) United States
(12) Patent Application Publication
(10) Pub. No.: US 2011/0027354 A1
(43) Pub. Date: Feb. 3, 2011

(54) ANTI-PARKINSONIAN COMPOUNDS

(76) Inventor: Hyoung-Chun Kim, Chunchon (KR)

Correspondence Address: JHK LAW, P.O. BOX 1078, LA CANADA, CA, 91012-1078, US

(21) Appl. No.: 12/935,673

(22) PCT Filed: Mar. 31, 2009
(86) PCT No.: PCT/IB09/51335

§ 371 Date: Sep. 30, 2010

Related U.S. Application Data
(60) Provisional application No. 61/041,538, filed on Apr. 1, 2008.

(51) Int. Cl. A61K 31/137; A61P 25/16; A61K 9/48

(52) U.S. Cl. 424/451; 514/654

(57) ABSTRACT

The present application describes a composition comprising a neuroprotective effective amount of N-methyl-N-propynyl-2-phenylethylamine (MPPE).

### BACKGROUND OF THE INVENTION

1. Field of the Invention

The invention relates to a neuroprotective compound. The invention further relates to a compound used to treat a variety of neurological conditions, including Parkinson's disease or the symptoms of Parkinson's disease, and learning and memory impairment in Alzheimer's disease.

2. General Background and State of the Art

Parkinson's disease (PD) is one of the major neurodegenerative disorders (Watanabe et al., 2005). It is characterized by the tetrad of akinesia, rigidity, tremor at rest and postural instability (Eberhardt and Schulz J, 2003; Oida et al., 2006). PD is associated with a selective degeneration of dopaminergic neurons in the substantia nigra pars compacta of the midbrain, and consequent reduction in striatal dopamine level (Oertel and ElIgring, 1995; Geng et al., 2007; Oida et al., 2006). One of the pathologic hallmark of PD is α-Synuclein (Syn) aggregation in the form of Lewy bodies in dopaminergic neurons in the ventrolateral portion of the substantia nigra (Sidhu et al., 2004; Galvin, 2006). Studies of purified Syn have revealed its ability to interact with diverse molecules including monoamines. Monoamine metabolism is associated with oxidative conditions that may contribute to dopamine (DA)—Syn interactions promoting aggregation and neuronal damage (Galvin et al., 2006).

1-Methyl-4-phenyl-1,2,3,6-tetrahydropyridine (MPTP) has been well-known to produce neuropathological changes similar to those observed in PD (Araki et al., 2001). Therefore, MPTP has been used to produce animal model for Parkinsonian condition (Speciale et al., 2002). Monoamine oxidase-B (MAO-B)-mediated production of 1-methyl-4-phenylpyridinium (MPP+), an active metabolite of MPTP, is necessary for inducing neurotoxic effect. This leads to subsequent dopaminergic neuron death, increased free radical generation. (Yang et al., 2005). MAO-B activity within reactive microglia in PD degrades the neurotransmitter DA, and then forms H₂O₂ and toxic aldehyde metabolites of DA (Nagatsu and Sawada, 2006; Mandel et al., 2005). H₂O₂ produces highly toxic reactive oxygen species (ROS) by Fenton reaction (Fe⁺²+H₂O₂→—Fe⁺³+.OH+OH⁻) (Budni et al., 2007) that is catalyzed by Fe²⁺ or Cu⁺ (Nagatsu and Sawada, 2006). It has been suggested that iron- or Fenton reaction-induced oxidative stress may play a critical role in the animal model for neurodegenerative disease (Yang et al., 2005), including MPTP neurotoxicity (Speciale, 2002).

Treatment with MPTP reduced levels of the brain-derived neurotrophic factor (BDNF) and glial cell line-derived neurotrophic factor (GDNF) in the nigrostriatal region of brain (Nagatsu and Sawada, 2005). The changes in those levels may be related to activated microglia in the DA neurons (Nagatsu and Sawada, 2005; Nagatsu et al., 2000). In addition, BDNF and GDNF may converge both at the phosphoinositide 3-kinase (PI3K)/Akt pathway (Sagi et al. 2006; Schober et al., 2007).

Selegiline, a therapeutic agent of Parkinson's disease, is a selective irreversible MAO-B inhibitor ich has antioxidant- and neuroprotective-effects (Takahata et al., 2005; Budni, 2007). Since dopamine is metabolized mainly by MAO-B in the brain, selegiline increases dopamine content in the central nervous system (Heinonen and Lammintausta, 1991). Recent studies suggest that neuroprotection in laboratory models may be related to the capacity of selegiline to up-regulate a series of antioxidant, which promotes cell survival, and that selegiline also reduces oxidative stress caused by catabolism of dopamine (Budni, 2007). In spite of the therapeutic potential of selegiline, its clinical application has been limited, because of its metabolism to d-amphetamine and methamphetamine (MA) (Am et al., 2004).

Therefore, we examined N-methyl-N-propynyl-2-phenylethylamine (MPPE), a selegiline analog, which may not be metabolized to d-amphetamine and methamphetamine (MA), on the MPTP-induced dopaminergic toxicity. We, then, evaluate the behavioral changes after repeated treatment with MPPE to understand whether MPPE induces behavioral side effects as shown in selegiline case. It was examined effects of MPPE on the striatal changes in the oxidative stress and neurotrophic factors in mice. The results suggest that MPPE attenuates MPTP-induced toxicity with guaranteed safety profile.

### SUMMARY OF THE INVENTION

Parkinson's disease (PD) is characterized by relatively selective nigrostriatal dopaminergic degeneration. 1-Methyl-4-phenyl-1,2,3,6-tetrahydropyridine (MPTP) is well known to damage the nigrostiatal dopaminergic neuron as seen in Parkinson's disease.

Selegiline, a selective monoamine oxidase-B inhibitor, has been used for the therapy of PD. It possesses antioxidant effects on the central nervous system. In spite of the neuroprotective effect of selegiline, clinical approach of selegiline, has been limited because of its metabolism to d-amphetamine and methamphetamine (MA). Since N-methyl-N-propenyl-2-phenylethylamine (NIPPE), a selegiline analog does not show behavioural side effects as shown in selegiline case, it was examined whether MPPE prevents MPTP-induced dopaminergic neurotoxicity.

MPTP-induced reductions in the locomotor activity and rota-rod performance were significantly attenuated in the presence of selegiline or MPPE. This attenuation was more pronounced in the MPPE-pretreated mice than selegiline-pretreated case.

Pretreatment with MPPE, or selegiline significantly attenuated MPTP-induced reductions in the nigral tyrosine hydroxylase-immunoreactivity (TH-IR), TH activity, dopamine level. In addition, MPTP-induced decreases in the brain derived neurotrophic factor and glial cell line-derived neurotrophic factor, phosphoinositide 3-kinase and phospho-Akt at Ser473 were attenuated in the presence of selegiline or MPPE. On the other hand, MPTP-induced increases in the microgliosis as labeled by F4/80 and alpha-synuclein expression were attenuated in the presence of selegiline or MPPE. These findings were more evidenced in the MPPE-treated case than selegiline-treated case.

These results indicate that MPPE exerts anti-Parkinsonian effects with safe profile, and that MPPE-mediated anti-inflammatory and neurotrophic actions are essentials in response to MPTP insult.

In one aspect, the invention is directed to a composition comprising a neuroprotective effective amount of N-methyl-N-propynyl-2-phenylethylamine (MPPE) or an analog thereof or a physiologically acceptable salt thereof together with a pharmaceutical carrier or excipient. The composition may be in sustained release dosage form. The composition is directed a Parkinson's disease symptom treatment effective amount.

In another aspect, the invention is directed to a unit dosage formulation for treatment of Parkinson's disease, comprising the composition described above or a pharmaceutically acceptable salt thereof in a form that is designed for oral ingestion by humans, wherein the N-methyl-N-propynyl-2-phenylethylamine (MPPE) or an analog or salt thereof is present at a dosage which renders the N-methyl-N-propynyl-2-phenylethylamine (MPPE) or an analog thereof therapeutically effective in substantially reducing symptoms of Parkinson's disease, without causing unacceptable side effects. The unit dosage formulation may include a digestible capsule. In one aspect, the dosage of the N-methyl-N-propynyl-2-phenylethylamine (MPPE) or an analog thereof may be about 250 milligrams/day or less.

In another aspect, the invention is directed to a method of treating symptoms of Parkinson's disease comprising administering to a patient or animal in need of such treatment an effective anti-Parkinsonism amount of the composition described above. The composition may be in sustained release dosage form. The composition may also comprise a neuroprotective agent. The composition may include a digestible capsule, and may be administered at about 250 milligrams/day or less.

In still another aspect, the invention is directed to a method of preventing decrease of dopamine production in substantia nigra of a subject comprising administering to the subject a protective effective amount of the composition described above.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGS. 1A-1B show structure of selegiline (A) and synthesis of T-methyl-N-propynyl-2-phenylethylamine (MPPE; B).

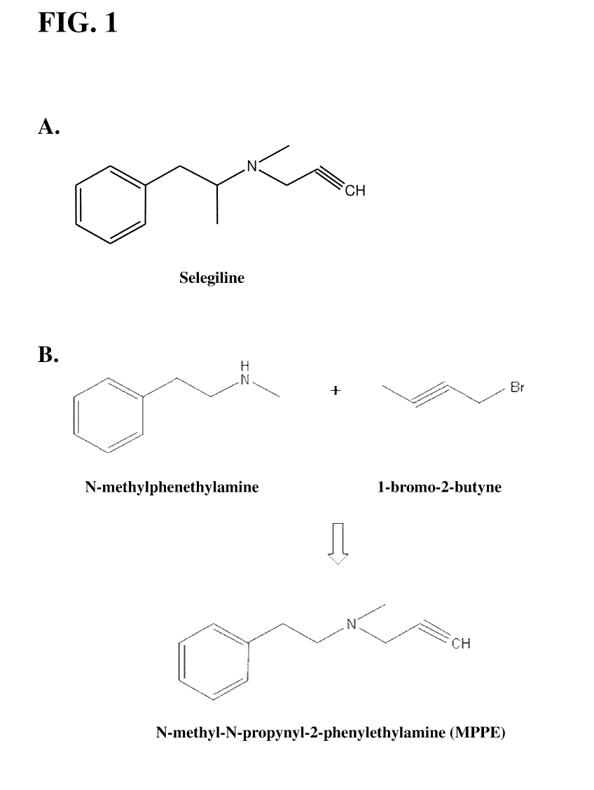

FIGS. 2A-2C show changes in the locomotor activities (A), locomotor tracing patterns (B) and conditioned place preference (CPP; C) induced by prolonged treatment with saline, selegiline (Sel), MPPE or methamphetamine (MA) in mice. Each value is the mean±S.E.M. of 5 animals. ^{a}p<0.05 vs. saline, ^{b}p<0.01 vs. saline, ^{c}p<002 vs, either dose of selegilne, ^{d}p<0.01 vs. either dose of selegilne, ^{e}p<0.01. MA 0.5 mg/kg, ^{f}p<0.01 vs. MA 1.0 mg/kg, ^{g}p<0.05 vs. selegiline 2.5 mg/kg and ^{h}p<0.01 vs. selegiline 5.0 mg/kg (ANOVA with Fisher's PLSD test). Note typical circling locomotor patterns as shown in the treatment with Sel or MA (drug control). However, treatment with MPPE did not show these behavioural side effects.

FIG. 3 shows experimental protocol for examing effects of MPPE 2.5 mg/kg/day 10, i.p.) or selegiline (Sel 2.5 mg/kg/day 10, i.p.) on the MPTP (25 mg/kg/day 7, s.c.)-induced dopaminergic toxicity.

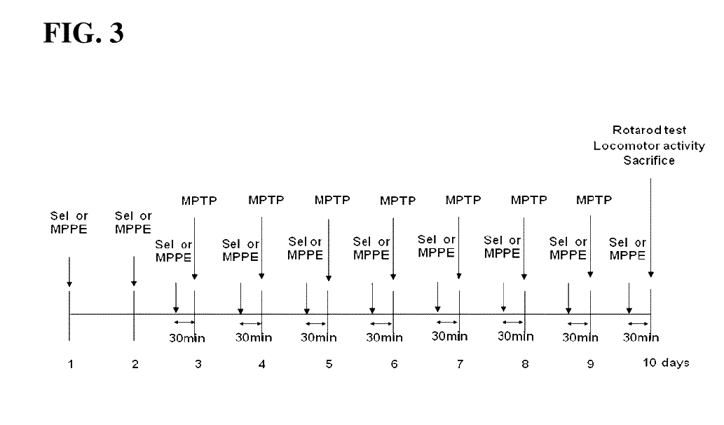

FIGS. 4A-4B show effect of selegiline (Sel) or MPPE on the MPTP-induced behavioral impairments [reduced locomotor activity and its pattern (A) and rota-rod performance (B)]. Each value is the mean±S.E.M. of 5 animals. ^{a}p<0.05 vs. saline+saline, ^{b}p<0.01 vs. saline+saline, ^{c}p<0.05 vs. saline+MPTP, ^{d}p<0.01 vs. saline+MPTP, ^{e}p<0.01 vs. selegiline+MPTP and ^{f}p<0.05 vs. selegiline+MPTP (ANOVA with DMR test).

FIGS. 5A-5D show elket of selegiline (Sel) or MPPE on the MPTP-induced striatal decreases in the dopamine (DA; A), 3,4-dihydroxyphenylacetic acid (DOPAC; B), homovanillic acid (HVA; C) and DA turnover rate (D) in the mice. Each value is the mean±S.E.M. of 6 animals. ^{a}p<0.01 vs. Saline+Saline, ^{b}p<005 vs. Saline+MPTP, ^{c}p<0.01 vs. Saline+MPTP and ^{d}p<0.05 vs. Selegiline+MPTP (ANOVA followed by Fischer's PLSD test).

FIGS. 6A-6C show effect of selegiline (Sel) or MPPE on the MPTP-induced striatal decrease in tyrosine hydroxylase (TH)-like immunoreactivity (TH-IR) [immunocytochemistry for TH (A), western boltting analysis for pan-TH, TH phospho-ser 19-like immunoreactivity (TH phospho-ser19-IR), TH phospho-ser31-like immunoreactivity (TH phospho-ser31-IR), and TH phospho-ser40-like immunoreactivity (TH phospho-ser40-IR) (B)] and activity of TH (C) in mice. Each value is the mean S.E.M. of 6 mice, ^{a}p<0.01 vs, saline+saline, ^{b}p<0.05 vs. saline+MPTP, ^{c}p<0.01 vs. salin+MPTP, ^{d}p<0.05 vs. selegiline+MPTP and ^{e}p<0.05 vs. selegiline+MPTP (ANOVA with Fischer's PLSD test).

FIGS. 7A-7C show effect of selegiline (Sel) or MPPE on the MPTP-induced nigral decreases in tyrosine hydroxylase immunoreactivity (TH-IR) [immunocytochemistry for TH (A), western blotting analysis for pan-TH, phospho-ser19-like immunoreactivity (TH phospho-ser19-IR), TH phospho-ser31-like immunoreactivity (TH phospho-ser31-IR), and TH phospho-ser40-like immunoreactivity (TH phospho-ser40-IR) (B)] and activity of TH (C) in the mice. Each value is the mean S.E.M. of 6 mice, ^{a}p<0.01 vs. saline+saline, ^{b}p<0.05 vs. saline+MPTP, ^{c}p<0.01 vs. saline+MPTP and ^{d}p<0.05 vs. selegiline MPTP (ANOVA with Fischer's PLSD test).

FIGS. 8A-8C show effects of selegiline (Sel) or MPPE on the MPTP-induced formation of reactive oxygen species (ROS; A) and expressions of the protein carbonyl (B), F4/80 (C) and oligornergic α-Synuclein (C) in the striatum of the mice. Each value is the mean S.E.M. of 6 mice. ^{a}p<0.05 vs. Saline+Saline, ^{b}<0.01 vs. Saline+Saline, ^{c}p<0.05 vs. Saline+MPTP, ^{d}p<0.01 vs. Saline+MPTP and ^{e}p<0.05 vs. Selegiline+MPTP (ANOVA with Fischer's PLSD test).

FIGS. 9A-9B show effect of selegiline (Sel) or MPPE on the MPTP-induced striatal decreases in the brain derived neurotrophic factor-like immunoreactivity (BDNF-IR), glial cell line-derived neurotrophic factor-like immunoreactivity (GDNF-IR) (A), phospho-Akt-like immunoreactivity (p-Akt-IR) and phospho-phosphoinositide 3-kinase-like immunoreactivity (p-PI3K-IR) (B) mice. Each value is the mean S.E.M. of 4 mice, ^{a}p<0.01 vs. saline+saline, ^{b}p<0.05 vs. saline+MPTP, ^{c}p<0.01 vs. saline+MPTP, ^{d}p<0.05 vs. selegiline+MPTP and ^{e}P<0.01 vs. selegiline+MPTP (ANOVA with Fischer's PLSD test).

### DETAILED DESCRIPTION OF THE PREFERRED EMBODIMENTS

In the present application, “a” and “an” are used to refer to both single and a plurality of objects.

As used herein, “effective amount” is an amount sufficient to effect beneficial or desired clinical or biochemical results. An effective amount can be administered one or more times. For purposes of this invention, an effective amount of a selegiline analog compound is an amount that is sufficient to palliate, ameliorate, stabilize, reverse, slow or delay the progression of a disease state or condition. In a preferred embodiment of the invention, the “effective amount” is defined as an amount of compound capable of preventing decrease in formation of dopamine in substantia nigra, and is an amount that substantially reduces the symptoms of Parkinson's disease. Other forms of effective amount may be for the treatment or prevention of the learning or memory impairment related to Alzheimer's disease. In yet another embodiment, the “effective amount” is defined as the neuroprotective effective amount of the selegiline, analog compound.

As used herein, administration “in combination with” one or more further therapeutic agents includes simultaneous (concurrent) and consecutive administration in any order.

As used herein, “mammal” or “subject” for purposes of treatment refers to any animal classified as a mammal, including humans, domestic and farm animals, and zoo, sports, or pet animals, such as dogs, cats, cattle, horses, sheep, pigs, and so on. Preferably, the mammal is human.

As used herein, “neuroprotective” agent refers to drugs or chemical agents intended to prevent damage to the brain or spinal cord from ischemia, stroke, convulsions, or trauma. Some must be administered before the event, but others may be effective for some time after. They act by a variety of mechanisms, but often directly or indirectly minimize the damage produced by endogenous excitatory amino acids. Neuroprotection also includes protection against neurodegeneration and neurotoxins. Further, by “neuroprotective” it is meant to include intervention that slows or halts the progression of neuronal degeneration. Neuroprotection may also be used for prevention or progression of a disease if it can be identified at a presymptomatic stage.

As used herein, “Parkinson's disease” refers to a chronic progressive nervous disease chiefly of later life that is linked to decreased dopamine production in the substantia nigra. Symptoms include stooped posture, resting tremor, weakness of resting muscles, a shuffling gait, speech impediments, movement difficulties and an eventual slowing of mental processes and dementia.

As used herein, “N-methyl-N-propynyl-2-phenylethylamine (MPPE) analog” may be any variant of MPPE that has an anti-Parkinsonian effect and is not metabolized to d-amphetamine and metamphetamine. The MPPE analog attenuates MPTP-induced toxicity with guaranteed safety profile, without showing behavioral side effects associated with administration of selegiline.

Therapeutic Formulations

Administration of the MPPE compound and its analogs and their mixtures and/or pharmaceutically acceptable salts can be orally or transdermally or by intravenous, intramuscular, subcutaneous, intrathecal, epidural or intracerebro-ventricular injection. Effective dosage levels can vary widely, e.g., from about 0.25 to about 250 mg/day, but actual amounts will, of course, depend on the state and circumstances of the patient being treated. As those skilled in the art recognize, many factors that modify the action of the active substance herein will be taken into account by the treating physician such as the age, body weight, sex, diet and condition of the patient, the time of administration, the rate and route of administration, and so forth. Optimal dosages for a given set of conditions can be ascertained by those skilled in the art using conventional dosage determination tests in view of the experimental data provided herein.

Therapeutic compositions containing the UTE compound and its analogs, their mixtures and/or pharmaceutically acceptable salts will ordinarily be formulated with one or more pharmaceutically acceptable ingredients in accordance with known and established practice. Thus, the MPPE compound and its analogs, their mixtures and/or pharmaceutically acceptable salts can be formulated as a liquid, powder, elixir, injectable solution, etc. Formulations for oral use can be provided as hard gelatin capsules wherein the MPPE compound and its analogs, their mixtures and/or pharmaceutically acceptable salts are mixed with an inert solid diluent such as calcium carbonate, calcium phosphate or kaolin, or as soft gelatin capsules wherein the MPPE compound and its analogs, their mixtures and/or pharmaceutically acceptable salts are mixed with an oleaginous medium, e.g., liquid paraffin or olive oil.

Aqueous suspensions can contain the MPPE compound and its analogs, their mixtures and/or pharmaceutically acceptable salts in admixture with pharmaceutically acceptable excipients such as suspending agents, e.g., sodium carboxymethyl cellulose, methylcellulose, hydroxypropylmethylcellulose, sodium alginate, polyvinylpyrrolidone, gum tragacanth and gum acacia; dispersing or wetting agents such as naturally occurring phosphatide, e.g., lecithin, or condensation products of an alkaline oxide with fatty acids, e.g., polyoxyethylene stearate, or condensation products of ethylene oxide with long chain aliphatic alcohols, e.g, heptadecaethylene-oxycetanol, or condensation products of ethylene oxide with partial esters derived from fatty acids and a hexitol, polyoxyethylene sorbitol monoleate or condensation products of ethylene oxide with partial esters derived from fatty acids and hexitol anhydrides, e.g., polyoxyethylene sorbitan monoleate. Such aqueous suspensions can also contain one or more preservatives, e.g., ethyl-or-n-propyl-p-hydroxy benzoate, one or more coloring agents, one or more flavoring agents and one or more sweetening agents, such as sucrose, saccharin or sodium or calcium cyclamate.

Dispersible powders and granules suitable for preparation of an aqueous suspension by the addition of water provide the MPPE compound and its analogs, their mixtures and/or pharmaceutically acceptable salts in admixture with a dispersing or wetting agent, suspending agent and one or more preservatives. Suitable dispersing or wetting agents and suspending agents are exemplified by those already mentioned above. Additional excipients, sweetening, flavoring and coloring agents, can also be present. Syrups and elixirs can be formulated with sweetening agents, for example glycerol, sorbitol or sucrose. Such formulations can also contain a demulcent, a preservative and flavoring and coloring agents.

The MPPE compound and its analogs, their mixtures and/or pharmaceutically acceptable salts are advantageously provided in sustained release dosage form of which many kinds are known, e.g., as described in U.S. Pat. Nos. 4,788,055; 4,816,264; 4,828,836; 4,834,965; 4,834,985; 4,996,047; 5,071,646; and, 5,133,974, the contents of which are incorporated by reference herein.

It is also within the scope of this invention to administer the MPPE compound and its analogs, their mixtures and/or pharmaceutically acceptable salts prior to, concurrently with, or after administration of any other known pharmacologically active agent useful for treating or treating the symptoms of Parkinson's disease. Such pharmacologically active agents may include without limitation other neuroprotective agents.

Neuroprotective agents attempt to save ischemic neurons in the brain from irreversible injury. Other neuroprotective agents prevent potentially detrimental events associated with return of blood flow. Although return of blood flow to the brain is generally associated with improved outcome, reperfusion may contribute to additional brain injury. Returning blood contains leukocytes that may occlude small vessels and release toxic products. Ischemia leads to excessive activation of excitatory amino acid receptors, accumulation of intracellular calcium, and release of other toxic products that cause cellular injury. By preventing excitatory neurotransmitter release, neuroprotective agents may reduce deleterious effects of ischemia on cells.

Instructions

The present invention is also directed to instructions regarding the use the inventive MPPE compound and its analogs, for treating a variety of neurological conditions, including Parkinson's disease or the symptoms of Parkinson's disease, learning and memory impairment in Alzheimer's disease. Such instructions may be in a permanent or temporary format. The instructions may be in written form, such as but not limited to a textbook, protocol book, catalog, internee web site and so on. Such instructions may be in relation to but not limited to the sale and use of the MPPE compound and its analogs. The instructions may be presented via a computer screen on a cathode ray tube, LCD, LED, and so on, so long as the instructions are visible through the eye. The instructions may also be in the form of audio/visual media, or as part of a kit for treating the various symptoms as indicated above.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Materials and Methods

### Example 1.1

### Animal

All mice were treated in strict accordance with the NIH Giude for the 1 mane Care and Use of Laboratory Animals (NTH Guide for the Care and Use of Laboratory Animals). C57BL/6.1 mice weighing about 25±3 g were maintained on a 12 h/12 h light/dark cycle and fed ad libitum. They were adapted for 2 weeks to the above conditions before experimentation.

### Example 1.2

### Synthesis of Analog

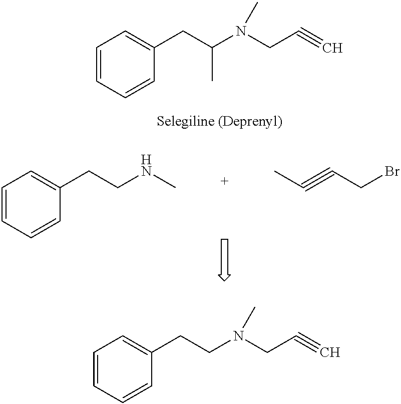

### N-methyl-N-propyn phenylethyl (MPPE)

### Example 1.3

### Drug Treatments

Selegiline or MPTP was injected (2.5 or 5 mg/kg, i.p.) once a day for 7 consecutive days. Methamphetamine, a positive control, was also administered (0.5 or 1 mg/kg, i.p.) once a day for 7 days.

Selegiline (2.5 mg/kg, i.p.) or MPPE (2.5 mg/kg, i.p.) was administered once a day from day 1 to day 10. MPTP was daily injected (25 mg/kg, s.c.) 30 min after selegiline- or MPPE-treatment from day 3 to day 9.

### Example 1.4

### Conditioned Place Preference (CPP)

For conditioned place preference (CPP) test, mice received an i.p. injection of saline just before entering the white or black compartment. MA (0.5 or 1.0 mg/kg, i.p.), selegiline (2.5 or 5.0 mg/kg, i.p.), and MPPE (2.5 or 5.0 mg/kg, i.p.) dissolved in saline was administered immediately before the mice were placed in the white compartment. On day 1, the mice were pre-exposed to the test apparatus for 15 minutes. The guillotine style doors were raised and mice were allowed to move freely between the two compartments. On day 2, the time each mouse spent in each compartment was recorded for 15 minutes. On days 3, 5, 7, 9, 11, and 13, the mice were injected with each drug before being confined to the white compartment, the non-preferred side, for 40 minutes. On day 14, the guillotine doors were raised. The mice were initially placed in the tunnel and the time spent by the mice in the two compartments was recorded for 15 minutes. The scores were calculated from the differences in the time spent in the white compartment in the testing and pre-testing phases. Data were analyzed between 09:00 and 17:00 hours.

### Example 1.5

### Locomotor Activity

Locomotor activity measured for 30 min one day after the last MPTP administration using an automated video-tracking system (Noldus Information Technology, Wagenin, The Netherlands). Eight test boxes (40×40×30 cm high) were operated simultaneously by an IBM computer. Animals were studied individually during locomotion in each test box, where they were adapted for 5 min before starting the experiment. A printout for each session showed the pattern of the ambulatory movements of the test box. The distance traveled in cm by the animals in horizontal locomotor activity was analyzed. Data were collected and analyzed between 09:00 and 17:00 h (Kim et al., 2001).

### Example 1.6

### Rota-rod Test

The apparatus (Ugo Basile model 7650) consisted of a base platform and a rotating rod with a nonslippery surface. The rod was placed at a height of 15 cm from the base. The rod, 30 cm in length, was divided into 5 equal sections by 6 opaque disks (so that the subjects cannot be distracted by one another). To assess motor performance, the mice were first trained on the apparatus 2 minutes at a rate 4 r.p.m. per 30 s prior to the test. The test was performed 30 minutes after training and an accelerating paradigm was applied at a rate 4 r.p.m. per 30 s, starting from 4 r.p.m. to a maximum speed of 40 r.p.m., then the rotation speed was kept constant at 40 r.p.m. for a maximum of 300 s. The duration for which the animal could maintain balance on the rotating drum was measured as the rotarod latency, with a maximal cut-off time of 300 s.

### Example 1.7

### Immunocytochemistry

### Example 1.7.1

### Histology

Animals were sacrified at 1 day after MPTP-treatment. They were anesthetized with 60% urethane and perfused transcardially with 200 ml of 50 mM phosphate buffered saline (PBS), followed by 50 ml of paraformaldehyde in PBS. The brain were fixed at 4° C. for 24 h in the same fixative and then cryoprotected in 30% sucrose. The brains were sectioned on a horizontal sliding microstome into 35 μm transverse free-floating sections.

### Example 1.7.2

### Immunocytochemistry

The immunocytochemistry was performed as described previously (Kim et al., 2000a; Kim et al., 2000b). Briefly, prior to incubation with the primary antibodies, sections were preincubated with 0.3% hydrogen peroxide in PBS for 30 min (to block endogenous peroxidase activity), then in PBS containing 0.4% Triton X-100 for 20 min and 1% no: mal serum for 20 min. After a 48 h incubation with the primary antibody at 4° C., sections were incubated with the secondary biotinylated antisera (1:1000 dilution; Vector, Brulingame, Calif.) for 1 h, washed, and immersed in avidin-biotin-peroxidase complex (ABC Elite kit, Vector) for 1 hr. Sections were always washed three times with PBS between each incubation step. 3,3′-Diaminobenzidine (DAB) was used as the chromogen. The quantitative analyses were performed using a computer-based image analysis system (Optimas version 6.2; Neurolucida Program) (Kim et al., 1999).

### Example 1.8

### Western Blot

The western blot assays were performed as described previously (thong et al., 1997). Tissues were homogenized in lysis buffer, containing 200 mM Tris HCl (pH 6.8), 1% SDS, 5 mM ethylene glycol tetraacetic acid, 5 mM ethylenediaminetetraacetic acid, 10% glycerol, 1× phosphatase inhibitor cocktail I, 1× protease inhibitor cocktail. The supernatant fraction was subsequently centrifuged at 30,000×g for 30 min. The resulting pellet was resuspended in the sample buffer. Proteins (20-50 ug/lane) were separated by 6%, 8%, 10% or 15% sodium dodecyl sulfate-polyacrylamide gel electrophoresis and transferred onto the nitrocellulose membranes. Following transfer, the nitrocellulose membranes were preincubated with 5% non-fat milk and incubated overnight at 4° C. with anti-β-actin (Sigma, 1:50000), anti-TH (Chemicon, 1:5000), anti-TH phosphoser19 (Chemicon, 1:5000), anti-TH phosphoser31 (Chemicon, 1:500), anti-TH phosphoser40 (Chemicon, 1:500), anti-F 4/80 (Serotec, 1:500), anti-BDNF (Chemicon, 1:500), anti-GDNF (Santa-Cruz, 1:250), anti-Akt (Cell signaling, 1:1000), anti-phospho Akt ser 473 (Cell signaling, 1:1000), anti-PI3K (Cell signaling, 1:1000), anti-phospho PI3K (Cell signaling, 1:500) and anti-α-synuclein (BD Transduction, 1:500) antibody. After incubation with primary antibody, membranes were incubated with the secondary anti-rabbit IgG, Horseradish Peroxidase (1:1000 dilution, Amersham) or anti-mouse IgG, Horseradish Peroxidase (1:1000, Sigma), or anti-goat IgG, Horseradish Peroxidase (1:1000, Sigma) for 2 h, washed. Subsequently visualized with the Amersham ECL system (Amersham, Arlington Heights, Ill., USA).

### Example 1.9

### HPLC Analysis

At 1 day after last MPTP injections mice were killed by cervical dislocation, the brains were removed and placed on an ice-cooled plate. Striatum was dissected and immediately frozen on dry ice and store at −70° C. until extraction. Brain regions obtained from each animal were weighed, ultrasonicated in 10% perchloric acid containing 10 ng/mg of the internal standard dihydroxybenzilamine, and centrifuged at 20,000 g for 10 min. The levels of DA and its metabolites 3,4-dihydroxyphenylacetic acid (DOPAC) and homovanillic acid (HVA) in brain tissue extracts were determined by HPLC coupled with electrochemical detection as described (Ali et al., 1994). Briefly, striatal tissues were sonicated in 0.2 M perchloric acid (20% W/V) containing the internal standard 3,4-dihydroxybenzylamine (10 mg wet tissue/ml) The homogenate was centrifuged and a 20-μl aliquot of the supernatant was injected into the HPLC equipped with a 3-μm C18 column. The mobile phase was comprised of 26 ml of acetonitrile, 21 ml of tetrahydrofuran and 960 ml of 0.15 M monochloroacetic acid (pH 3.0) containing 50 mg/l of EDTA and 200 mg/l of sodium octyl sulfate. The amount of DA, DOPAC and HVA were determined by comparison of peak height ratio of tissue sample with standards, and were expressed in nanograms per gram of wet weight of tissue.

### Example 1.10

### TH Activity

TH activity was measured according to the method of Lucock et al. with some modification (Duan et al., 2005). Briefly, cells (1×10⁶/mil) were washed by PBS and lysed in 400 μL TH working solution (1-tyrosine: 300 μmol/L; FeSO4: 1 mmol/L; NaAc: 200 μmol/L; NSD-1050: 500 μmol/L; DTT: 1 mmol/L; MES: 40 mmol/L, pH 5.2-5.6) with freezing-thawing repeatedly for three times. The cell lysate was reacted for 3 h at 25° C. The reaction was stopped by 0.4 mol/L perchloric acid, and then the cell solution was centrifuged at 14,000×g, 4× for 10 min. Supernatants were collected to assay the amounts of 1-dopa by HPLC-ECD. The activity of TH was expressed as that amount of 1-dopa per minute and per cell.

### Example 1.11

### Determination of ROS Formation

The extent of reactive oxygen species (ROS) formation in the prefrontal cortex was assessed by measuring the conversion from 2′,7′-dichlorofluorescin diacetate (DCFH-DA) to dichlorofluoresin. (DCF) as describe by Bourre et al. with slight modification (Sutsumi, S et al., 2002). Brain homogenates were added to a tube containing 2 ml of PBS with 10 nmole of DCFH-DA, dissolved in methanol. Mixtures were incubated at 37° C. for 3 h and then measured the absorbance at 480 nm excitation and 525 nm emission. DCF is used as a standard.

### Example 1.12

### Determination of Protein Carbonyl (Oxyblot Assay)

Total protein (15 mg) was used to perform the Oxyblot assay. The amount of oxidized proteins was measured using the Oxyblot kit (Chemicon International, CA), following the manufacturer's instructions. Briefly, the protein carbonyl content was measured by first forming labeled protein hydrazone derivatives using 2,4-dinitrophenylhydrazide (DNP). The INP-derivatized protein samples were separated by polyacrylamide gel electrophoresis followed by Western blotting. Blots were then incubated with primary antibody specific to the DNP moiety, followed by incubation with a horseradish peroxidase-antibody conjugate directed against the primary antibody. The blots were then treated with chemiluminescent reagents (Amersham) (Gemma et al., 2004).

### Example 1.13

### Statistics

Statistical significance was analyzed by one-way ANOVA. Post-hoc Fischer's PLSD test was followed for the comparison among groups. P values<0.05 were deemed to be statistically significant.

### Example 2

### Results

### Example 2.1—Behavior Evaluation

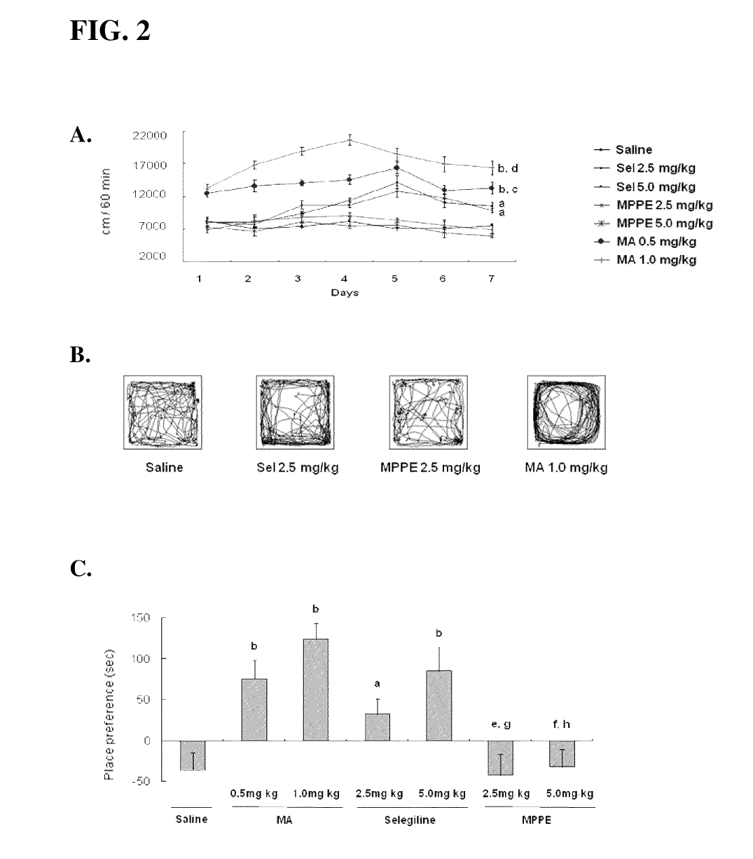

Saline-treated mice exhibited basal locomotor activities. Repeated treatment with selegiline (2.5 or 5.0 mg/kg/i.p./day×7) (selegiline 2.5 or 5.0 mg/kg/i.p. vs. saline, p<0.05) or MA (0.5 or 1.0 mg/kg/i.p./day×7) (MA 0.5 or 1.0 mg/kg/i.p. vs. saline, p<0.01; MA 0.5 mg/kg/i.p. vs. either dose of selegiline, p<0.02; MA 1.0 mg/kg/i.p. vs. either dose of selegiline, p<0.01), significantly increased locomotor activities with circling locomotor patterns. However, treatment with MPPE (2.5 or 5.0 mg/kg/i.p./day×7) did not significantly alter locomotor activities. Locomotor patterns mediated by MPPE were comparable to those by saline (FIGS. 2A, 2B). In addition, treatment with selegiline (selegiline 2.5 mg/kg/i.p. vs. saline, p<0.05; Selegiline 5.0 mg/kg/i.p. vs. saline, p<0.01) or MA (MA 0.5 or 1.0 mg/kg/i.p. vs. saline, p<0.01) significantly increased compared with saline in the conditioned place preference (CPP) test. However, treatment with MITE (MPPE 2.5 mg/kg/i.p. vs. MA 0.5 mg/kg, p<0.01, MPPE 2.5 mg/kg/i.p. vs. selegiline 2.5 mg/kg, p<:0.05; MPPE 5.0 mg/kg/i.p. vs. MA 1.0 mg/kg, p<0.01, MPPE 5.0 mg/kg/i.p. vs. selegiline 5.0 mg/kg, p<0.01) did not significantly change (FIG. 2C).

### Example 2.2

### Effects of MPPE or Selegiline on the Hypolocomotion Induced by MPTP

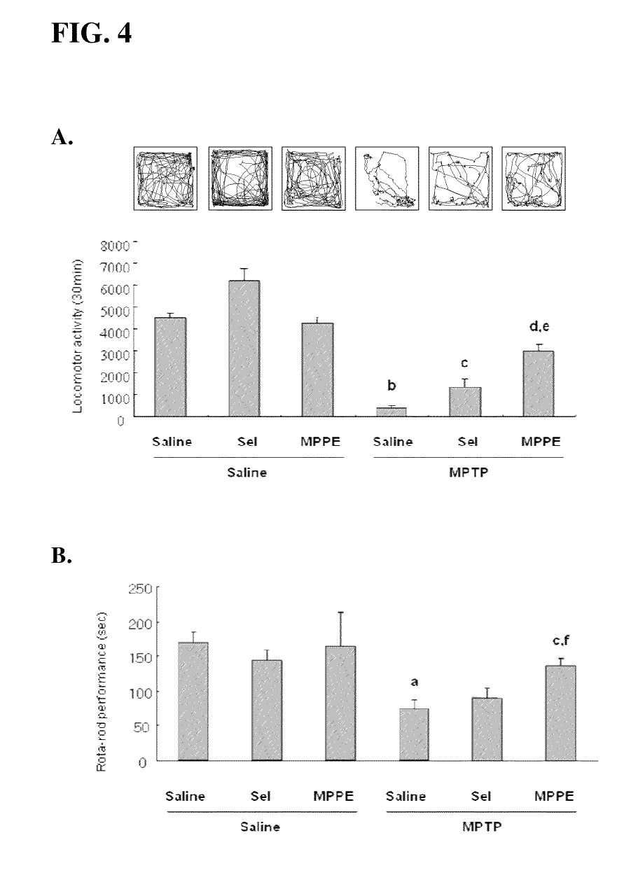

Effects of MPPE or selegiline on the reductions in the locomotor activity and its tracing pattern in MPTP-treated mice were examined. MPTP-treated mice showed significant hypolocomotor activity (p<0.01 vs. saline), which was attenuated by the treatment with selegiline (2.5 mg/kg/i.p./day×9) (p<0.05 vs. MPTP alone) or MPPE (2.5 mg/kg/i.p./day×9) (p<0.01 vs. MPTP alone). This attenuating effect was more pronounced in the MPPE-pretreated mice (p<0.01 vs, selegiline+MPTP) than those in the selegiline-pretreated mice (FIG. 4A).

MPTP treatment impaired Rota-rod performance in mice (p<0.05 vs. saline). This impairment was significantly attenuated by MPPE (p<0.05 vs. MPTP alone, p<0.05 vs. selegiline+MPTP), but not by selegiline (FIG. 4B).

### Example 2.3

### Effects of MPPE or Selegiline on the Dopaminergic Losses Induced by MPTP

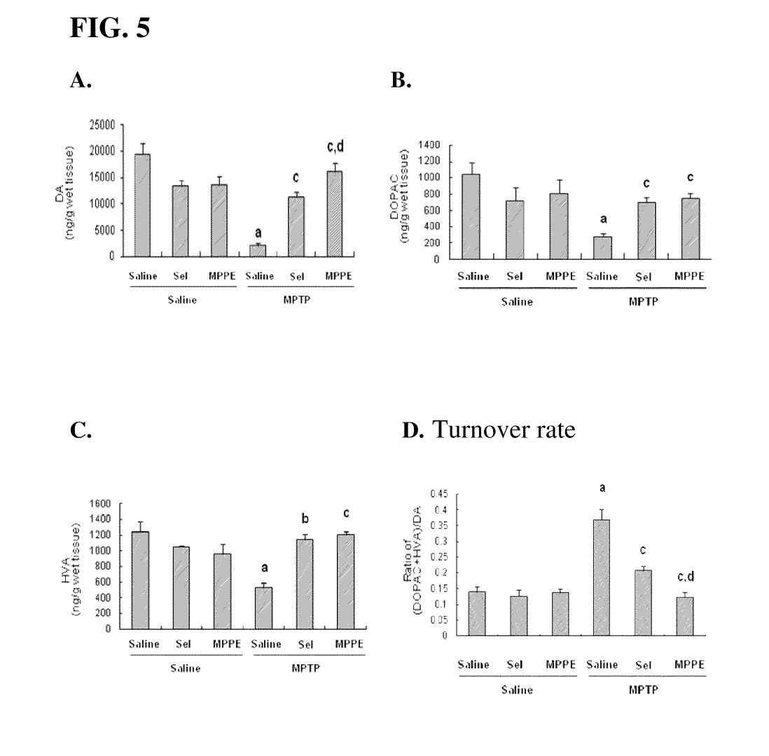

Either selegiline or MPPE alone did not show any significant effect on the levels of DA, DOPAC, HVA, TH activity, and TH-immunoreactivity. MPTP administration resulted in significant reductions in the contents of dopamine (DA) (p<0.01 vs, saline its metabolites 3,4-dihydroxyphenylacetic acid (DOPAC) (p<0.01 vs. saline), and homovanillic acid (HVA) (p<0.01 vs. saline) in the striatum of the mice. These reductions were attenuated with the treatment with selegiline (DA: p<0.01 vs. MPTP alone; DOPAC: p<0.01 vs. MPTP alone; HVA: p<0.05 vs. MPTP alone), and with MPPE (DA: p<0.01 vs. MPTP alone; DOPAC: p<0.01 vs. MPTP alone; HVA: p<0.01 vs. MPTP alone). This attenuation for DA level was more pronounced in the MPPE-treated mice (p<0.05 vs. selegiline+MPTP) than selegiline-treated mice (FIGS. 5A, 5B, 5C). In addition, DA turnover rate was significantly increased in MPTP-treated mice (p<0.01 vs. saline). This increase was attenuated in the presence of selegiline (p<0.01 vs, MPTP alone) or MPPE (p<0.01 vs. MPTP alone). This attenuation by MPPE (p<0.05 vs. selegiline MPTP) was more evident than by selegiline (FIG. 5D).

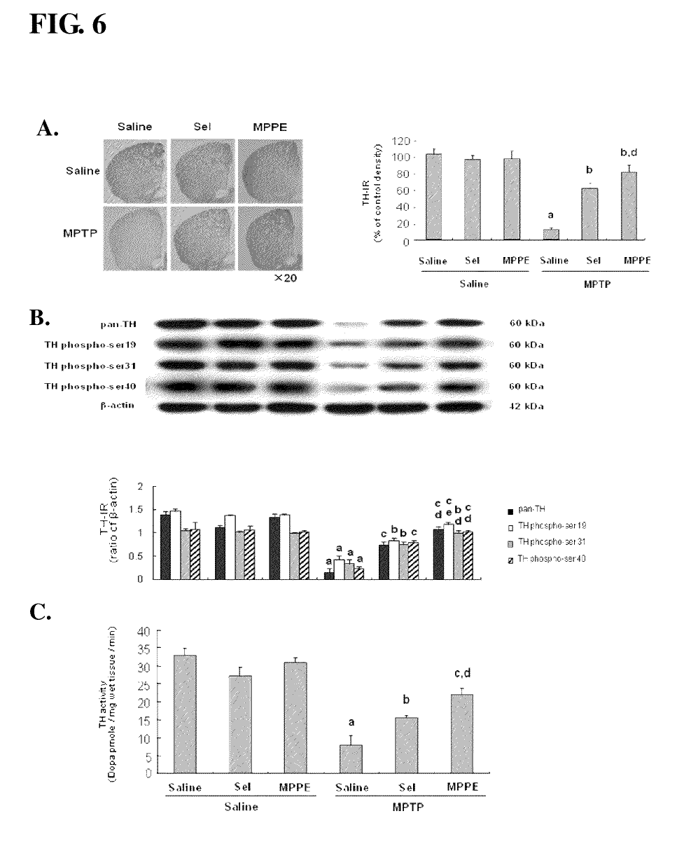

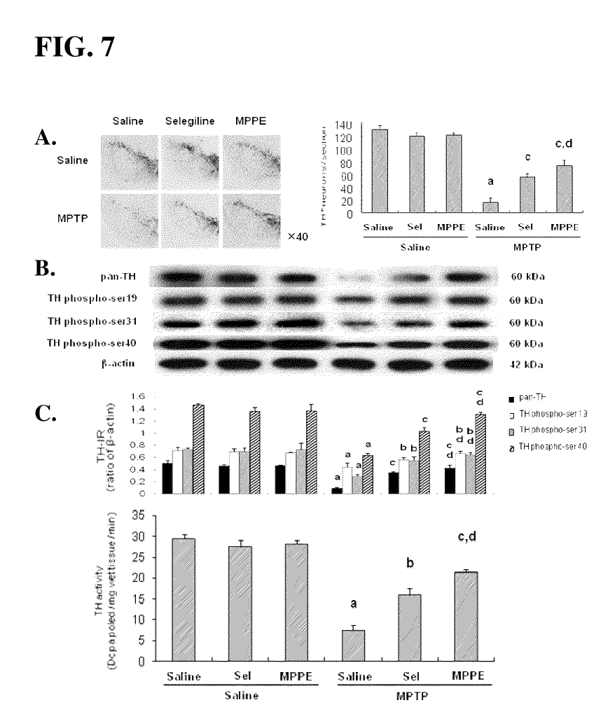

Consistently, TH activity was significantly decreased one day after final MPTP treatment [striatum: p<0.01 vs. saline; substantia nigra (SN): p<0.01 vs. saline]. This decrease was attenuated by the treatment with selegiline (striatum: p<0.05 vs, MPTP alone; SN: p<0.05 vs. MPTP alone) or by the MPPE (striatum: p<0.01 vs. MPTP alone; SN: p<0.01 vs. MPTP alone). MPPE-mediated attenuation was more effective (striatum: p<0.05 vs. selegiline+1 MPTP; SN: p<0.05 vs. selegiline+MPTP) than selegiline case (FIGS. 6C, 7C). In addition, repeated injection with MPTP significantly decreased TH-immunoreactivity (TH-IR), as evaluated by immunocytochemistry (ICC) (striatum: p<0.01 vs. saline; SN: p<0.01 vs, saline) (FIGS. 6A, 7A) and by western blotting (striatum: p<0.01 vs. saline; SN: p<0.01 vs. saline) (FIGS. 6B, 7B). This decrease was attenuated in the presence of selegiline (striatum: p<0.05 vs. MPTP alone in the ICC, p<0.01 vs. MPTP alone in the western blotting; SN: p<0.01 vs. MPTP alone in both ICC and western blotting) or MPPE (striatum: p<0.05 vs. MPTP alone in the ICC, p<0.01 vs. MPTP alone in the western blotting; SN: p<0.01 vs. MPTP alone in both ICC and western blotting). This attenuation was more evident (striatum: p<0.05 vs. selegiline MPTP in the ICC and western blotting; SN: p<0.05 vs. selegiline+MPTP in the ICC and western bating) in the MPPE treatment than in the selegiline (FIGS. 6A, 6B, 7A, 7B).

TH phosphorylation (TH phosphorylation at Ser¹⁹ Ser³¹ and Ser⁴⁰) was also significantly decreased after MPTP treatment in the striatum (TH phosphorylation at Ser¹⁹: p<0.01 vs, saline; TH phosphorylation at Ser³¹: p<0.01 vs. saline; TH phosphorylation at Ser⁴⁰: p<0.01 vs. saline) (FIG. 6B) and SN (TH phosphorylation at Ser¹⁹: p<0.01 vs. saline; TH phosphorylation at Ser³¹: p<0.01 vs. saline; TH phosphorylation at Ser⁴⁰: p<0.01 vs. saline) (FIG. 7B) of mice. This phenomenon was also reversed by pre-treatment with selegiline (striatum: p<0.05 vs. MPTP alone in the TH phosphorylation at Ser¹⁹ and TH phosphorylation at Ser³¹, p<0.01 vs. MPTP alone in the TH phosphorylation at Ser⁴⁰; p<0.05 vs. MPTP alone in the TH phosphorylation at Ser¹⁹ and TH phosphorylation at Ser³¹, p<0.01 vs. MPTP alone in the TH phosphorylation at Ser⁴⁰) or by MPPE (striatum: p<0.01 vs. MPTP alone in the TH phosphorylation at Ser¹⁹ and TH phosphorylation at Ser⁴⁰, p<0.05 vs. MPTP alone in the TH phosphorylation at Ser³¹; SN: p<0.05 vs. MPTP alone in the TH phosphorylation at Ser¹⁹ and TH phosphorylation at Ser³¹, p<0.01 vs. MPTP alone in the TH phosphorylation at Ser⁴⁰) in the striatum (FIG. 6B) and in the SN (FIG. 7B). Effect of MPPE on the MPTP-induced impairments in dopaminergic system was more effective (striatum: p<0.01 vs. selegiline±MPTP in the TH phosphorylation at Ser¹⁹, p<0.05 vs, selegiline+MPTP in the TH phosphorylation at Ser³¹ and TH phosphorylation at Ser40; SN: p<0.05 vs. selegiline MPTP in the TH phosphorylation at Ser¹⁹ phosphorylation at Ser³¹ and TH phosphorylation at Ser⁴⁰) than selegiline (FIGS. 6B, 7B).

### Example 2.4

### Effects of MPPE or Selegiline on the F4/80 Expression Induced by MPTP

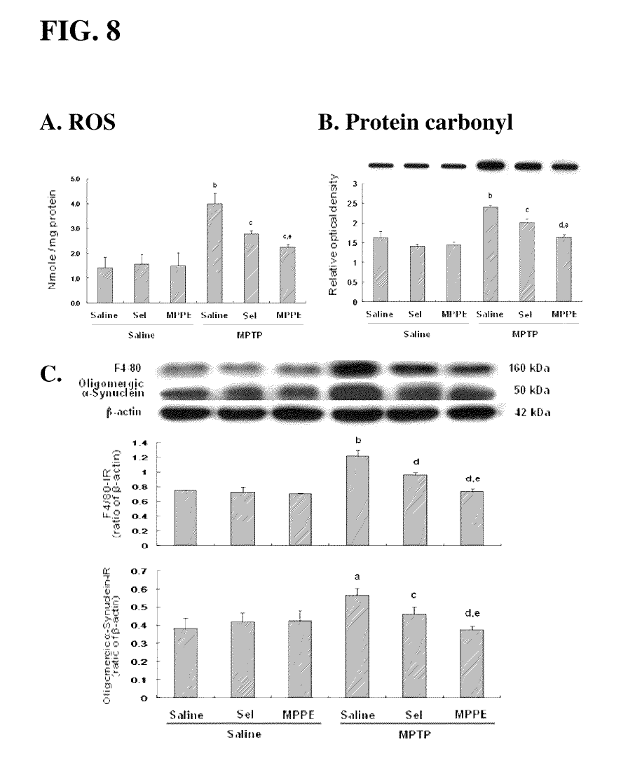

Protein expression of F4/80, a marker of reactive microglia, was barely induced in the absence of MPTP. F4/80-like immunoreactivity was significantly increased in the striatum of MPTP-treated mice (p<0.01 vs. saline). Pre-treatment with selegiline (p<0.01 vs. MPTP alone) or MPPE (p<0.01 vs. MPTP alone) significantly blocked increase in F4/80-immunoreactivity induced by MPTP (FIG. 8C).

### Example 2.5

### Effects of MPPE or Selegiline on the MPTP-induced Oxidative Stress and Oligomergic α-synuclein Expression

The striatal changes in the oxidative stress markers, such as reactive oxygen species (ROS) (FIG. 8A) and protein carbonyl (Oxyblot assay) (FIG. 8B), were evaluated one day after final MPTP administration. Either selegiline or MPPE alone exhibited a little induction in ROS level and protein carbonyl expression. MPTP-induced increases in the ROS level (p<0.01 vs. saline), and protein carbonyl expression (p<0.01 vs. Saline) were observed. These increases were attenuated with the treatment with selegiline (ROS: p<0.05 vs. MPTP alone; protein carbonyl: p<0.05 vs. MPTP alone) or MPPE (ROS: p<0.05 vs, MPTP alone; protein carbonyl: p<0.01 vs. MPTP alone). This attenuating effect appeared to be more underlined in the MPPE-pretreated mice (ROS: p<0.05 vs. selegiline+MPTP; protein carbonyl: p<0.05 vs. selegiline MPTP) than in the selegiline-treated mice (FIGS. 8A, 8B).

Oligomergic α-synuclein was significantly increased in the striatum of MPTP-treated mice (p<0.05 vs. saline), which was significantly attenuated in the presence of selegiline (p<0.05 vs. MPTP alone) or MPPE (p<0.01 vs. MPTP alone). MPPE-pretreated mice exerted more protective effect (p<0.05 vs. selegiline MPTP) than selegiline-pretreated mice (FIG. 8C).

### Example 2.6

### Effects of MPPE or Selegiline on the MPTP-induced BDNF and GDNF

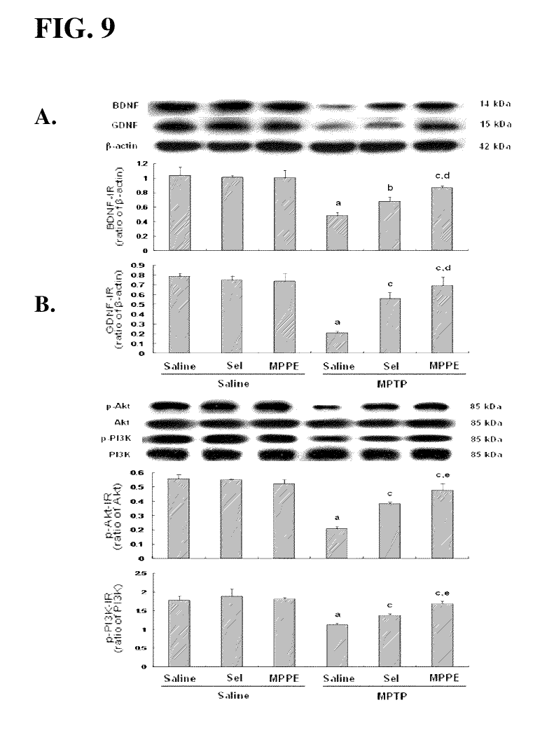

The striatal protein expressions of the brain derived neurotrophic factor (BDNF) and glial cell line-derived neurotrophic factor (GDNF) were examined by western blot. In the absence of MPTP, high levels of the BDNF and GDNF were expressed. Treatment with MPTP significantly reduced in the expressions of BDNF (p<0.01 vs. Saline) and GDNF (p<0.01 vs. Saline), which were significantly attenuated by pre-treatment with selegiline (BDNF: p<0.05 vs. MPTP alone; GDNF: p<0.01 vs. MPTP alone) or MPPE (BDNF: p<0.01 vs. MPTP alone; GDNF: vs. MPTP alone). MPPE-pretreated mice revealed more effective (BDNF: p<0.05 vs. selegiline+MPTP; GDNF: p<0.05 vs. selegiline+MPTP) in enhancing these expressions than selegiline-pretreated mice (FIG. 9A).

### Example 2.7

### Effects of MPPE or Selegiline on the MPTP-induced Changes in the Phosphorylation of Akt at Ser473 and Phosphorylation of Phosphoinositide 3-kinase (PI3K)

High levels of phospho-Akt and phospho-PI3K expressions were observed in the striatum of the mice in the absence of MPTP. The significant decreases in the phospho-Akt (p<0.01 vs. saline) and phospho-PI3K (p<0.01 vs. saline) were observed in the striatum of MPTP-treated mice, while total-Akt and total-PI3K were not affected. These decreased in the phospho-Akt and phospho-PI3K were significantly attenuated by selegiline (phospho-Akt: p<0.01 vs. MPTP alone; phospho-PI3K: p<0.01 vs. MPTP alone) or MPPE (phospho-Akt: p<0.01 vs. MPTP alone; phospho-PI3K: p<0.01 vs. MPTP alone). These attenuations in the phospho-Akt and phospho-PI3K expressions were more pronounced in MPPE-treated mice (phospho-Akt: p<0.01 vs. selegiline+MPTP; phospho-PI3K: p<0.01 vs. selegiline+MPTP) than in selegiline-treated mice (FIG. 9B).

### REFERENCES

Albanese A, Granata R, Gregori B, Piccardi P, Colosimo C and Tonali P. Chronic administration of 1-methyl-4-phenyl-1,2,3,6-tetrahydropyridine to monkeys: behavioural, morphological and biochemical correlates. Neuroscience 1993; 55: 823-832.
Ali S F, Newport G D, Holson R R, Slikker W, Jr and Bowyer J. Low environmental temperatures or pharmacologic agents that produce hypothermia decrease methamphetamine neurotoxicity in mice. Brain Res 1994; 658: 33-38.
Am O B, Amit T and Youdim M B. Contrasting neuroprotective and neurotoxic actions of respective metabolites of anti-Parkinson drugs rasagiline and selegiline. Neurosci Lett. 2004; 355(3): 169-72.
Araki T, Kumagai T, Tanaka K, Matsubara M, Kato H, Itoyama Y and Imai Y. Neuroprotective effect of riluzole in MPTP-treated mice. Brain Res 2001; 918(1-2): 176-81.
Barcia C, Sanchez Bahillo Fernandez-Villalba E, Bautista V, Poza YPM, Fernandez-Barreiro A, Hursch E C and Herrero M T. Evidence of active microglia in substantia nigra pars compacta of parkinsonian monkeys 1 year after MPTP exposure. Glia 2004; 46: 402-409.
Budni P, de Lima M N, Polydoro M, Moreira J C, Schroder N and Dal-Pizzol F. Antioxidant effects of selegiline in oxidative stress induced by iron neonatal treatment in rats. Neurochem Res 2007; 32(6): 965-72. Epub 2007 Mar. 31.
Budni P, de Lima M N, Polydoro M, Moreira J C, Schroder N and Dal-Pizzo F. Antioxidant effects of selegiline in oxidative stress induced by iron neonatal treatment in rats. Neurochem Res 2007; 32(6): 965-72. Epub 2007 Mar. 31.
Cassarino D S, Parks J K, Parker W D and Bennett J P. The parkinsonian neurotoxin MPP+ opens the mitochondrial permeability transition pore and releases cytochrome c in isolated mitochondria via an oxidative mechanism. Biochem Biophys Acta 1999; 1453: 49-62.
Cohen G, Pasik P, Cohen B et al. Pargyline and deprenyl prevent the neurotoxicity of 1-methyl-4-phenyl-1,2,3,6-tetrahydropyridine (MPTP) in monkeys, Eur Pharmacol 1984; 106: 209-210.
Czlonkowska A, Kohutnicka M, Kurkowska-Jastrzebska and Czlonkowski A. Microglial reaction in MPTP (1-methyl-4-phenyl-1,2,3,6-tetrahydropyridine) induced Parkinson's disease mice model Neurodegenration 1996; 5: 137-143.
Duan C L, Su Y, Zhao C L, Lu L L, Xu Q Y and Yang H. The assays of activities and function of TH, AADC, and GCH1 and their potential use in ex vivo gene therapy of PD. Brain Res Brain Res Protoc. 2005; 16(1-3): 37-43.
Ebadi M, Sharma S, Shavali S and Refaey H E. Neuroprotective actions of selegiline. J. Neurosci Res 2002; 67(3): 285-9.
Eberhardt O and Schulz J B. Apoptotic mechanisms and antiapoptotic therapy in the MPTP model of Parkinson's disease. Toxicol Lett 2003; 139: 135-51.
Gal S, Zheng H, Fridkin A and Youdirn M B H. Novel multifunctional neuroprotective iron chelator-monoamine oxidase inhibitor drags for neurodegenerative diseases, in vivo selective brain monoamine oxidase inhibition and prevention of MPTP-induced strata dopamine depletion. Journal of neurochemistry 2005; 95, 79-88.
Galvin J E Interaction of alpha-synuclein and dopamine metabolites in the pathogenesis of Parkinson's disease: a case for the selective vulnerability of the substantia nigra. Acta Neuropathol 2006; 112(2): 115-26. Epub 2006 Jun. 22.
Galvin J E. Interaction of alpha-synuclein and dopamine metabolites in the pathogenesis of Parkinson's disease: a case for the selective vulnerability of the substantia nigra. Acta Neuropathol 2006; 112(2): 115-26. Epub 2006 Jun. 22.
Gao H M, Jiang J, Wilson B, Zhang W, Hong J S and Liu B. Microglial activation-mediated delayed and progressive degeneration of rat nigral dopaminergic neurons: relevance to Parkinson's disease 2002; J. Neurochem. 2002; 81: 1285-1297.
Gash D M, Zhang Z, Ovadia A, Cass W A, Yi A, Simmerman L, Russell D, Martin D, Lapchak P A, Collins F, Hotter B J and Gerhardt G A. Fuctional recovery in parkinsonian monkeys treated with GDNF, Nature 1996 Mar. 21; 380(6571): 252-5.
Gemma C, Stellwagen H, Fister M, Coultrap S J, Mesches M H, Browning M D and Bickford P C. Rosiglitazone improves contextual fear conditioning in aged rats. Neuroreport 2004; 15(14): 2255-9.
Geng X, Tian X, Tu P and Pu X. Neuroprotective effects of echinacoside in the mouse MPTP model of Parkinson's disease. Eur J Pharmacol 2007; 564(1-3): 66-74. Epub 2007 Feb. 16.
Gerlach M, Riederer P and Youdim M B H. Molecular mechanisms for neurodegeneration: synergism between reactive oxygen species, calcium and excitotoxic amino acids. Adv. Neurol. 1996a; 69, 177-194.
Gerlach M, Youdim M B H and Riederer P. Pharmacology of selegiline, Neurology 1996b; 47(3); 137-145.
Hantraye P, Varastet M, Peschanski M, Riche D, Cesaro P, Willer J C a id Maziere M, Stable Parkinsonian syndrome and uneven loss of dopamine fibres following chronic MPTP administration in baboons. Neuroscience 1993; 53: 169-178.
Heinonen E H and Lammintausta R. A review of the pharmacology of selegiline. Acta Neurol Scand Suppl 1991; 136: 44-59.
Heinonen E H and Lammintausta R. A review of the pharmacology of selegiline, Acta Neurol Scand Suppl 1991; 136: 66-59.
Henchcliffe C, Schumacher H C and Burgut F T. Recent advances in Parkinson's disease therapy: use of monoamine oxidase inhibitors. Expert Rev Neurother 2005 November; 5(6): 811-21.
Kim H C, Bing O, Jhoo W K, Suh J H, Shin E J, Kato K and Ko K H. An immunocytochemical study of mitochondrial manganese superoxide dismutase in the rat hippocampus after kainate administration. Neurosci. Lett 2000a; 281: 65-68.
Kim H C, Bing G, Shin E J, Jhoo H S, Cheon M A, Lee S H, Choi K H, Kim J I and Jhoo W K. Dextromethorphan affects cocaine-mediated behavioral pattern in parallel with a long-lasting Fos-related antigen-immunoreactivity. Life Sci 2001; 69(6): 615-24.
Kim H C, Jhoo W K, Bing G, Shin E J, Wie M B, Kim W K and Kato K H. Phenidone prevents kainate-induced neurotoxicity via antioxidant mechanisms. Brain Res 2000; 874: 15-23.
Kim H C, Jhoo W K, Choi D Y, Im D H, Shin E J, Suh J H, Floyd R A and Bing G. Protection of methamphetamine nigrostriatal toxicity by dietary selenium Brain Res 1999; 851(1-2): 76-86.
Kobori N, Waymire J C, Haycock J W, Clifton G L and Dash P K. Enhancement of tyrosine hydroxylase phosphorylation and activity by glial line-derived neurotrophic factor, 0.1 Biol Chem 2004; 279(3): 2182-91. Epub 2003 Oct. 21.
Kwan E, Baker G B., Shuaib A, Ling L and Todd K G. N-methyl,N-propargyl-2-phenylethaylamine (MPPE), an analog of deprenyl, increases neuronal cell survival in thiamin deficiency encephalopathy. Drag Development Research 2000, 51, 244-252.
Leret M L, San Millan J A, Fabre E, Gredilla R and Baja G. Deprenyl protects from MPTP-induced Parkinson-like syndrome and glutathione oxidation in rat stratum, Toxicoligy 2002; 170, 165-171.
Magyar K, Szende B and Lengyel J et al. The pharmacology of B-type selective monoamine oxidase inhibitors; milestones in (−)-deprenyl research. J Neural Transm Suppl 1996; 48: 29-43.
Mandel S, Weinreb O, Amit T and Youdim M B. Mechanism of neuroprotective action of the anti-Parkinson drug rasagiline and its derivatives. Brain Res Brain Res Rev 2005; 48(2): 379-87.
Mount M P, Lira A, Grimes D, Smith P D, Faucher S, Slack R and Anisman H. Involvement of interferon-gamma in microglial-mediated loss of dopaminergic neurons. The Journal of neuroscience 2007; 27(12): 3328-3337.
Nagatsu T and Sawada M. Inflammatory process in Parkinson's disease: role for cytokines. Curr Pham in Des 2005; 11(8): 999-1016.
Nagatsu T and Sawada M. Molecular mechanism of the relation of monoamine oxidase B and its inhibitors to Parkinson's disease: possible implications of glial cells. J Neural Transm Suppl 2006; (71): 53-65.
Nagatsu. T, Mogi M, Ichinose H and Togari A. Changes in cytokines and neurotrophins in Parkinson's disease. J Neural Transco Suppl 2000; (60): 277-90.
Oertel W H and Ellgring H. Parkinson's disease-medical education and psychosocial aspects, Patient Educ Coons 1995; 26(1-3): 71-9.
Oida Y, Kitaichi K, Nakayama H, Ito Y, Fujimoto Y, Shimazawa M, Nagai H and Hara H. Rifampicin attenuates the MPTP-induced neurotoxicity in mouse brain. Brain Res 2006; 1082(1): 196-204. Epub 2006 Mar. 3
Pallhagen S, Heinonen E, Hagglund J et al. Selegiline slows the progression of the symptoms of Parkinson disease. Neurology 2006; 66(8): 1200-1206.
Reynolds A D, Glanzer J G, Kadiu I, Ricardo-Dukelow M, Chaudhuri A, Ciborowski P, Cerny R, Gelman B, Thomas M P, Mosley R L, and Gendelman H E. Nitrated alpha-synuclein-activated microglial profiling for Parkinson's disease, J Neurochem 2007; [Epub ahead of print].
Sagi Y, Mandel S, Amit T and Youdim M B. Activation of tyrosine kinase receptor signaling pathway by rasagiline facilitates neurorescue and restoration of nigrostriatal dopamine neurons in post-MPTP-induced parkinsonism. Neurobiol Dis 2007; 25(1): 35-44. Epub 2006 Oct. 20.
Schifitto G, Zhang J, Evans S R, Sacktor N, Simpson D, Millar L L, Hung V L, Miller E N, Smith E, Ellis R J, Valcour V, Singer F, Marra C M, Kolson D, Weihe J, Remmel R, Katzenstein D. Clifford DB; ACTG A5090 Team. A multicenter trial of selegiline transdermal system for HIV-associated cognitive impairment. Neurology 2007; 69(13): 1314-21. Epub 2007 Jul. 25.
Schober A, Peterziel H, von Bartheld C S, Simon H, Krieglstein K and Unsicker K. GDNF applied to the MPTP-lesioned nigrostriatal system requires TGF-beta for its neuroprotective action. Neurobiol Dis 2007; 25(2): 378-91. Epub 2006 Dec. 1.
Schweizer U. Bräuer A U, Köhrle J, Nitsch R and Savaskan N E. Selenium and brain function: a poorly recognized liaison. Brain Res Brain Res Rev 2004; 45(3); 164-78.
Sidhu A, Wersinger C, Moussa C E and Vernier P. The role of alpha-synuclein in both neuroprotection and neurodegeneration. Ann N Y Acad Sci 2004; 1035: 250-70.
Sidhu A, Wersinger C, Moussa C E and Vernier P. The role of alpha-synuclein in both neuroprotection and neurodegeneration. Ann N Y Acad Sci 2004; 1035: 250-70.
Speciale S G. MPTP: insights into parkinsonian neurodegeneration, Neurotoxicol Teratol 2002; 24(5): 607-20.
Speciale S G. MPTP: insights into parkinsonian neurodegeneration, Neurotoxicol Teratol 2002; 24(5): 607-20.
Sutsumi S, Akaike M, Arimitsu H, Imai H and Kato N. Circulating Corticosterone Alters the Rate of Neuropathological and Behavioral Changes Induced by Trimethyltin in Rats. Experimental Neurology 2002; 173(1): 86-94.
Szende B, Bokonyi Gy, Bocsi J, Keri Gy, Timar F and Magyar K. anti-apoptotic and apoptotic action of (−)-deprenyl and its metabolites, J Neural Transm 2001; 108: 25-33.
Takahata K, Shimazu S, Katsuki H, Yoneda F and Akaike A. Effects of selegiline on antioxidant systems in the nigrostriatum in rat. J Neural Transm 2006; 113(2): 151-8. Epub 2005 Jun. 15.
Tomac A, Lindqvist E, Lin L F, Ogren S O, Young D, Hoffer B J and Olson L. Protection and repair of the nigrostriatal dopaminergic system by GDNF in vivo. Nature 1995 Jan. 26; 373(6512): 335-9.
Watanabe Y, Himeda T and Araki T. Mechanisms of MPTP toxicity and their implications for therapy of Parkinson's disease. Med Sci Monit 2005; 11(1): RA17-23.
Yang L, Calingasan N Y, Chen J, Ley J J, Becker D A and Beal M F. A novel azulenyl nitrone antioxidant protects against MPTP and 3-nitropropionic acid neurotoxicities. Exp Neurol 2005; 191(1): 86-93.
Zhong G, Reis e Sousa C and Germain R N. Production, specificity, and functionality of monoclonal antibodies to specific peptide-major histocompatibility complex class II complexes formed by processing of exogenous protein. Proc Natl Acad Sci U S A 1997; 94(25): 13856-61.

All of the references cited herein are incorporated by reference in their entirely.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A composition comprising a neuroprotective effective amount of N-methyl-N-propynyl-2-phenylethylamine (MPPE) or an analog thereof or a physiologically acceptable salt thereof together with a pharmaceutically acceptable carrier or excipient.

2. The composition of claim 1 in sustained release dosage form.

3. The composition according to claim 1, comprising a Parkinson's disease symptom treatment effective amount.

4. A unit dosage formulation for treatment of Parkinson's disease, comprising the composition according to claim 1 or a pharmaceutically acceptable salt thereof in a form that is designed for oral ingestion by humans, wherein the N-methyl-N-propynyl-2-phenylethylamine (MPPE) or an analog or salt thereof is present at a dosage which renders the N-methyl-N-propynyl-2-phenylethylamine (MPPE) or an analog thereof therapeutically effective in substantially reducing symptoms of Parkinson's disease, without causing unacceptable side effects.

5. The unit dosage formulation of claim 4, comprising a digestible capsule, which encloses the N-methyl-N-propynyl-2-phenylethylamine (MPPE) or an analog thereof or pharmaceutically acceptable salt thereof.

6. The unit dosage formulation of claim 5, wherein the dosage of the N-methyl-N-propynyl-2-phenylethylamine (MPPE) or an analog thereof is about 250 milligrams/day or less.

7. A method of treating symptoms of Parkinson's disease comprising administering to a patient or animal in need of such treatment an effective anti-Parkinsonism amount of the composition according to claim 1.

8. The method of claim 7, wherein the composition is in sustained release dosage form.

9. The method of claim 8, wherein the composition further comprises a neuroprotective agent.
